(19) United States
(12) Patent Application Publication
(10) Pub. No.: US 2004/0192591 A1
(43) Pub. Date: Sep. 30, 2004

(54) Composition for treating Chlamydia infections and method for identifying same

(76) Inventors: Joseph L. Wooters, Brighton, MA (US); Benjamin J. Metcalf, Rochester, NY (US); Banumathi Sankaran, Pittsford, NY (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/722,695

(22) Filed: Nov. 24, 2003

Related U.S. Application Data
(60) Provisional application No. 60/428,585, filed on Nov. 22, 2002.

(51) Int. Cl. A61K 38/12; C12Q 1/18

(52) U.S. Cl. 514/9; 435/32

(57) ABSTRACT

The invention relates to therapeutic modalities and pharmaceutical compositions for the treatment of Chlamydia-infection using cyclophilin polypeptides and its corresponding human cellular binding partner or partners as a target for intervention. The present invention relates to the use of exogenous or engrafted sources of cyclophilins, anti-cyclophilin antibodies, cyclophilin decoys, soluble forms of cyclophilin-binding partners and small molecules which are supplied extracellularly, and act presumably by interrupting the binding of cyclophilin A with its cellular binding partner(s) or receptor(s), as a treatment for Chlamydia-infection. The present invention further relates to screening assays for the identification of compounds that inhibit the interaction of cyclophilin and its Chlamydia binding partners.

### RELATED APPLICATIONS

This application claims priority to U.S. Ser. No. 60/428,585, filed Nov. 22, 2002. The contents of this application are incorporated herein by reference in their entirety.

### FIELD OF THE INVENTION

The invention relates generally to vaccines and polypeptides and more particularly to a method for identifying Chlamydia polypeptides useful as vaccines against Chlamydia infections.

### BACKGROUND OF THE INVENTION

Members of the bacterial genus Chlamydia are obligate intracellular parasites of eukaryotic cells; these bacteria are unable to grow unless they are inside the eukaryotic cell. Chlamydia exist in two forms: (1) a nonreplicating, infectious particle called the elementary body (EB), which is released from ruptured infected cells and can be transmitted from one individual to another or from infected birds to humans; and (2) an intracytoplasmic form called the reticulate body (RB) that engages in replication and growth.

Multiple Chlamydia species have been reported to cause, or be associated with, disease. For example, Chlamydia psittaci infects a wide variety of birds and a number of mammals. Chlamydia trachomatis (C. trachomatis or CT), a bacterial species pathogenic to man, is the etiological agent of venereal lymphogranuloma (VLG), as well as trachoma, a chronic disease which is reported to affect more than 100 million people and can lead to blindness. This bacterium is also associated with various inflammatory pathologies of both male and female genitalia. Chlamydia pneumoniae (C. pneumonia TWAR organism) is associated with disease in humans.

### SUMMARY OF THE INVENTION

The invention is based in part on the discovery that cyclophilin polypeptides, which are eukaryotic in origin, are present in Chlamydia cells isolated from human cells. This finding has been used to develop novel methods for identifying Chlamydia polypeptide antigens that are useful as targets for developing therapeutic agents for treating Chlamydia infections. Also provided by the invention are methods of treating Chlamydia infections by administering agents that inhibit the expression or activity of a cyclophilin polypeptide.

In one aspect, the invention provides a method of identifying a cyclophilin-binding protein from a Chlamydia spp. by providing a cyclophilin polypeptide, contacting the cyclophilin polypeptide with a test agent, and determining whether the test agent binds the cyclophilin polypeptide. Binding of the test agent to the cyclophilin polypeptide indicates the test agent is a therapeutic target for treating a Chlamydia spp. infection.

In some embodiments, the cyclophilin is provided as a substantially purified cyclophilin polypeptide. Any cyclophilin polypeptide known in the art can be used, e.g., cyclophilin A, cyclophilin B, cyclophilin C, or cyclophilin D. As used herein, “substantially pure” polypeptide means a polypeptide separated from components that naturally accompany it. Typically, the polypeptide is substantially pure when it is at least 60%, by weight, free from the proteins and naturally-occurring organic molecules with which it is naturally associated. Preferably, the purity of the preparation is at least 75%, more preferably at least 90%, and most preferably at least 99%, by weight. A substantially pure cyclophilin polypeptide fragment can be obtained, for example, by extraction from a natural source, by expression of a recombinant nucleic acid encoding a cyclophilin polypeptide fragment, by expression of a cyclophilin polypeptide fragment fusion protein, or by chemical synthesis. A chemically synthesized polypeptide or a polypeptide produced in a cellular system different from the cell from which it naturally occurs is, by definition, substantially free from components that naturally accompany it. Accordingly, substantially pure polypeptides include those derived from eukaryotic organisms but synthesized in recombinant cells of E. coli or other prokaryotes. Purity can be measured by any appropriate methods, e.g., column chromatography, polyacrylamide gel electrophoresis, or by HPLC analysis.

The cyclophilin polypeptide can be provided as a labeled polypeptide. The label can be, e.g., a radioactive label or a non-radioactive label such as a fluorescent label. Alternatively, the cyclophilin can be labeled with a label such as biotin.

In some embodiments, the cyclophilin polypeptide is provided attached to a substrate. The substrate may include a plurality of cyclophilin polypeptides, e.g., one or more of cyclophilin A, cyclophilin B, cyclophilin C, cyclophilin D, or a mixture of these polypeptides. The cyclophilin polypeptides are provided on the substrate at one or more addressable locations.

The substrate can be, e.g., a planar surface or a spherical surface (such as a bead).

The method can also be performed by providing a cyclophilin polypeptide that is associated with a Chlamydia cell. The cyclophilin polypeptide can be provided as a labeled polypeptide. The label can be, e.g., a radioactive label or a non-radioactive label such as a fluorescent label. Alternatively, the cyclophilin can be labeled with a label such as biotin.

In some embodiments, the cyclophilin polypeptide is associated with a Chlamydia cell by binding to one or more Chlamydia polypeptides.

In some embodiments, the cyclophilin is provided in association with a Chlamydia elementary body.

Also provided by the invention is a method for identifying a therapeutic agent for treating a Chlamydia infection. The method includes providing a sample that includes a Chlamydia polypeptide and contacting the sample with a cyclophilin polypeptide under conditions allowing for formation of a complex between at least one Chlamydia protein in the sample and the cyclophilin polypeptide. The complex is then detected, and at least one Chlamydia protein in the complex is identified. The identified Chlamydia protein(s) is useful, e.g., as a target for identifying therapeutic agents for treating Chlamydia infections. For example, a cyclophilin-binding polypeptide can be used to raise antibodies that can, in turn, be used to treat Chlamydia infections. In some embodiments, the complex is detected with an anti-cyclophilin antibody.

Also provided by the invention is method for identifying a therapeutic agent for treating a Chiamydia infection by providing a sample that includes a Chlamydia polypeptide and a cyclophilin polypeptide and contacting the sample with a cyclophilin probe under conditions that allow for formation of a complex between the cyclophilin probe and the Chlamydia protein. The complex is detected and the Chlamydia protein in the complex is identified. The identified Chlamydia protein can be used, e.g., as a therapeutic target for treating a Chlamydia infection. For example, antibodies against the cyclophilin-binding Chlamydia protein can be raised and test for their efficacy in treating Chlamydia infection.

In some embodiments, the Chlamydia proteins are labeled, e.g., with biotin.

A cyclophilin probe is an agent that binds cyclophilin preferentially as a compared to binding to a non-cyclophilin molecule. A preferred cyclophilin probe is an anti-cyclophilin antibody.

The invention additionally provides a method of identifying a therapeutic agent for treating Chlamydia infections by providing a Chlamydia cell, contacting the cell with an agent that inhibits cyclophilin, and determining whether the agent inhibits the pathogenicity of the Chlamydia cell. Inhibition of pathogenicity of the Chlamydia cell indicates the agent is a therapeutic agent for treating Chlamydia.

In a further aspect, the invention provides a method of identifying an agent that inhibits infection of a eukaryotic host cell by a Chlamydia cell by providing a Chlamydia cell, contacting the cell with an agent that inhibits at least one activity of a cyclophilin polypeptide; and determining whether the agent inhibits infection of the Chlamydia cell.

In a still further aspect, the invention provides a method for identifying compounds capable of interfering with the formation of a complex between a Chlamydia cell and a cyclophilin polypeptide. The method includes: (a) producing a cyclophilin affinity fusion protein; (b) preincubating a compound with the cyclophilin affinity fusion protein of step (a); (c) adding Chlamydia to the incubate of step (b) under conditions which permit one or more Chlamydia proteins and the cyclophilin affinity fusion protein to form a complex; (d) contacting the incubate of step (c) with an affinity medium under conditions that allow the Chlamydia-cyclophilin affinity fusion protein complex to bind to the affinity medium; (e) determining the amount of the Chlamydia-cyclophilin affinity fusion protein complex formation by comparison to a control sample lacking the compound. Reduced binding of Chlamydia to the cyclophilin affinity fusion protein indicates the compound inhibits formation of the complex. The cyclophilin fusion polypeptide can be, e.g., cyclophilin A, cyclophilin B, cyclophilin C, or cyclophilin D. In some embodiments, the cyclophilin affinity fusion protein is a glutathione S-transferase-cyclophilin (GST-cyclophilin) fusion protein and/or the affinity medium includes glutathione-agarose beads.

In another aspect, the invention provides a method for identifying a compound capable of interfering with the formation of a complex between a cyclophilin and a Chlamydia affinity fusion protein. The method includes:(a) producing a Chlamydia affinity fusion protein; (b) preincubating a compound with the Chlamydia affinity fusion protein of step (a); (c) adding a cyclophilin to the incubate of step (b) under conditions which permit the cyclophilin and the Chlamydia affinity fusion protein to form a complex; (d) contacting the incubate of step (c) with an affinity medium under conditions that enable the cyclophilin-Chlamydia fusion protein complex to bind the affinity medium; and (e) determining the amount of the cyclophilin-Chlamydia affinity fusion protein complex formation by comparison to a control sample lacking the compound. Reduced binding indicates the compound inhibits cyclophilin-Chlamydia affinity fusion protein complex formation. The cyclophilin polypeptide can be, e.g., cyclophilin A, cyclophilin B, cyclophilin C, and cyclophilin D, or combinations thereof. In some embodiments, the cyclophilin affinity fusion protein is a glutathione S-transferase-cyclophilin (GST-cyclophilin) fusion protein and/or the affinity medium includes glutathione-agarose beads.

The cyclophilin can be labeled if desired with, e.g., a fluorescent label, a radioactive label, or a chemiluminescent label.

Also provided by the invention is a method of treating a Chlamydia infection in a subject by administering to the subject a therapeutically effective amount of an agent that inhibits activity of cyclophilin. In some embodiments, the agent is a polypeptide that includes a cyclophilin-binding region of an anti-cyclophilin antibody polypeptide. An example of such an agent is an anti-cyclophilin antibody. The antibody can be, e.g., a polyclonal antibody or a monoclonal antibody. The subject can be, e.g., a human, a non-human primate (such as a chimpanzee, gorilla, or new world monkey), cow, horse, pig, goat, sheep, dog, cat, or bird.

In other embodiments, the agent is an antibiotic that inhibits the activity of a cyclophilin polypeptide. An example of such an antibiotic is cyclosporin. The cyclosporin can be provided as a cyclosporin derivative.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention provides methods for identifying Chlamydia vaccine antigens, as well as compositions and methods using these antigens based on the ability of Chlamydia macromolecules (e.g., Chlamydia polypeptides) to interact with cyclophilin.

The invention is based in part on the discovery that human cyclophilin is present on the surface of Chlamydia elementary bodies (EB) recovered from human host cells. While not wishing to be bound by theory, it is believed that Chlamydia, an intracellular pathogen, sequesters cyclophilin polypeptides from its eukaryotic host in order to effect a productive infection of the host. It is additionally envisioned that a Chlamydia cell uses cyclophilin to gain entry into a new host cell during a subsequent round of infection. Thus, inhibitors of cyclophilin (such as an anti-cyclophilin antibody) can be used to inhibit infection of a eukaryotic host cell by a Chlamydia cell.

The invention is also based, in part, on the observation that cyclophilin, which is a very soluble protein, is present on the hydrophobic surface of EBs. While not wishing to be bound by theory, it is believed that a Chlamydia-derived protein(s) specifically interact with cyclophilin and anchor cyclophilin to the EB surface. Anti-Chlamydia agents can be therefore be designed based on the ability of a test agent to disrupt the association between a cyclophilin polypeptide and the EB.

Chlamydia Isolates and Culture Methods

In general, any Chlamydia strain can be used in the herein described methods. Examples of suitable Chlamydia strains include C. trachomatis, C. pittachis, and C. pneumoniae (TWAR).

Chlamydia spp. can be cultured using suitable host cells and methods known in the art. Such methods are described in, e.g., Stamm et al., Detection of Chlamydia trachomatis inclusions in McCoy cell cultures with fluorescein-conjugated monoclonal antibodies, J Clin Microbiol, 17:666-68. (1983).

Cyclophilin Polypeptides

Cyclophilin polypeptides are a class of proteins that bind to cyclosporin A (CsA). CsA is a fungal cyclic undecapeptide useful as an immunosuppressive therapeutic agent to prevent graft rejection. Cyclophilins demonstrate peptidyl-prolyl cis-trans isomerase (PPiase) or rotamase activity (Fischer, et al., Nature, 337:476-78, 1989). This enzyme catalyzes the cis-trans isomerization of proline-imidic peptide bonds in oligopeptides and has been demonstrated to accelerate the refolding of several proteins, including collagens (Bachinger, J. Biol. Chem., 262:17144-48, 1987).

The cyclophilins are apparently ubiquitous throughout nature, and the sequences of more than 27 cyclophilins and 17 FKBPs are known. Sequence alignment of the cyclophilin sequences reveals two “signature” sequences which are present in almost all of the cyclophilins (see Trandinh et al., FASEB J., 6:3410 1992)). In addition, the position corresponding to residue 121 in human cyclophilin A has been implicated in strong cyclosporin binding (see Liu et al., Biochem. 30:2306-2310 (1991)).

Cyclophilin A is a member of the “immunophilin family”, i.e., a group of related cellular factors involved in regulating immunity. Accordingly, several different cyclophilin polypeptides, or immunophilin family members, can be used in the methods of the present invention. Cyclophilin A is a 19 KD protein, which is abundantly expressed in a wide variety of cells. Cyclophilin A binds cyclosporin A, and possesses peptidyl-prolyl cis-trans isomerase (PPIase), and protein folding or “chaperone” activities. Four mammalian cyclophilins, which include cyclophilins A, B, C, and hCyP3, are described in Friedman et al., Proc. Natl. Acad. Sci. (USA) 90:6815-6819, 1993. A homologous protein is found in mouse macrophages, MAC-2 binding protein Chicheportiche et al., Proc. Natl. Acad. Sci., 269:5512-5577, 1994.

Unless otherwise indicated, the term “cyclophilin binding partner” as used herein means any Chlamydia cellular binding protein, cell surface binding protein, extracellular receptor, or intracellular binding protein, which has cyclophilin specific binding activity.

Examples of exogenous forms of cyclophilin include but are not limited to human cyclophilin A, other human cyclophilins, the cyclophilins of other species (i.e., mouse, yeast) derivatized cyclophilin, recombinant cyclophilin, cyclophilin fusion proteins or peptide fragments expressing the domain of cyclophilin which binds to its host cell receptor.

A further embodiment of the invention relates to the use of exogenous cyclophilin binding protein(s) to inhibit the interaction between cyclophilin and its cellular binding partner. Examples of exogenous forms of cyclophilin binding partners or receptors include, but are not limited, to derivatized cyclophilin binding partners, recombinant cyclophilin binding partners, fusion proteins or peptide fragments expressing the domain of the cyclophilin binding partner or receptor which binds to cyclophilin.

In a further embodiment, a therapeutic modality for Chlamydia infection embodying the genetic engineering of cells to produce or release cyclophilin constitutively or in response to suitable inducers and the supply of said cells to a patient by engraftment of exogenously transfected cells or treatment of the patient by gene therapy to transfect endogenous cell populations is envisioned. This therapeutic modality is likewise suitable to implement Chlamydia treatment by expression and release of cyclophilin mutants, molecular decoys or other mimics of cyclophilin that inhibit Chlamydia infection, for instance by interfering with the interactions between cyclophilin and such Chlamydia binding proteins, therefore, as are required for productive infection. Therapeutic modalities capitalizing on the genetic engineering of cells to express and release soluble forms of cyclophilin-binding proteins or the cyclophilin-binding domains thereof will likewise find utility in the practice of the present invention and are contemplated hereunder.

Cyclophilin may be derivatized by a variety of methods known to those skilled in the art. In a preferred embodiment of the invention, cyclophilin is pegylated by reacting cyclophilin with methoxypolyethylene glycol-succinimidyl succinate and 4-dimethylamino pyridine in methylene chloride with stirring for two days at room temperature in the dark. To block any unreacted sites, ethanolamine is added and the mixture incubated at room temperature with stirring for another 24 hours. The pegylated cyclophilin is purified from the reaction mixture by normal phase HPLC.

The recombinant forms of cyclophilin, the fusion proteins or peptides expressing the domain of cyclophilin that binds to its cellular receptor can be produced by synthetic techniques or via recombinant DNA technology using techniques well known in the art. Thus, methods for preparing the differentially expressed cyclophilin polypeptides and peptides of the invention are described herein. First, the polypeptides and peptides of the invention can be synthesized or prepared by techniques well known in the art e.g., Creighton, 1983, “Proteins: Structures and Molecular Principles”, W. H. Freeman and Co., N.Y., which is incorporated herein by reference in its entirety. Peptides can, for example, be synthesized on a solid support or in solution.

Alternatively, recombinant DNA methods, which are well known to those skilled in the art, can be used to construct expression vectors containing differentially expressed cyclophilin protein coding sequences and appropriate transcriptional/translational control signals. These methods include, for example, in vitro recombinant DNA techniques, synthetic techniques and in vivo recombination/genetic recombination e.g., the techniques described in Sambrook et al., 1989, Molecular Cloning: A Laboratory Manual, Cold Spring Harbor Press, Cold Spring Harbor, N.Y., which are incorporated by reference herein in their entirety, and Ausubel, 1989, supra. Alternatively, RNA capable of encoding differentially expressed cyclophilin protein sequences can be chemically synthesized using, for example, synthesizers e.g., the techniques described in “Oligonucleotide Synthesis”, 1984, Gait, M. J. ed., IRL Press, Oxford, which is incorporated by reference herein in its entirety.

A variety of host-expression vector systems can be utilized to express the cyclophilin coding sequences of the invention. Such host-expression systems represent vehicles by which the coding sequences of interest can be produced and subsequently purified, but also represent cells which can, when transformed or transfected with the appropriate nucleotide coding sequences, exhibit or produce and release the cyclophilin protein of the invention in situ. These include but are not limited to microorganisms such as bacteria (e.g., E. coli, B. subtilis) transformed with recombinant bacteriophage DNA, plasmid DNA or cosmid DNA expression vectors containing cyclophilin protein coding sequences; yeast (e.g., Saccharomyces, Pichia) transformed with recombinant yeast expression vectors containing the cyclophilin protein coding sequences; insect cell systems infected with recombinant virus expression vectors e.g., baculovirus containing the cyclophilin protein coding sequences; plant cell systems infected with recombinant virus expression vectors, e.g., cauliflower mosaic virus, CaMV; tobacco mosaic virus, TMV or transformed with recombinant plasmid expression vectors e.g., Ti plasmid containing cyclophilin protein coding sequences; or mammalian cell systems e.g. COS, CHO, BHK, 293, 3T3 harboring recombinant expression constructs containing promoters derived from the genome of mammalian cells e.g., metallothionein promoter, or from mammalian viruses e.g., the adenovirus late promoter; the vaccinia virus 7.5K promoter.

In bacterial systems, a number of expression vectors can be advantageously selected depending upon the use intended for the cyclophilin protein being expressed. For example, when a large quantity of such a protein is to be produced, for the generation of antibodies or to screen peptide libraries, vectors that direct the expression of high levels of fusion protein products that are readily purified are desirable. Such vectors include, but are not limited, to the E. coli expression vector pUR278 (Ruther et al., EMBO J. 2:1791, 1983), in which the cyclophilin protein coding sequence can be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

In an insect system, Autographa californica nuclear polyhidrosis virus (ACNPV) is used as a vector to express foreign genes. The virus grows in Spodoptera frugiperda cells. The cyclophilin coding sequence can be cloned individually into non-essential regions i.e., the polyhedrin gene of the virus and placed under control of an AcNPV promoter e.g. the polyhedrin promoters. Successful insertion of cyclophilin coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed, (e.g., see Smith et al., 1983, J. Viol. 46:584; Smith, U.S. Pat. No. 4,215,051).

In mammalian host cells, a number of expression systems, including viral-based expression systems, can be utilized and either the protein expressed on the cells or the cells expressing the protein may be utilized in accordance with the present invention. In cases where an adenovirus is used as an expression vector, the cyclophilin coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome e.g., region E1 or E3 will result in a recombinant virus that is viable and capable of expressing cyclophilin protein in infected hosts, See Logan & Shenk, Proc. Natl. Acad. Sci. USA 81:3655-3659, 1984. Specific initiation signals can also be required for efficient translation of inserted cyclophilin coding sequences. These signals include the ATG initiation codon and adjacent sequences. In cases where an entire cyclophilin gene, including its own initiation codon and adjacent sequences, is inserted into the appropriate expression vector, no additional translational control signals can be needed. However, in cases where only a portion of the cyclophilin coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See Bittner et al., Methods in Enzymol. 153:516-544, 1987).

In addition, a host cell strain can be chosen which modulates the expression of the inserted sequences, or modifies and processes the gene product in the specific fashion desired. Such modifications, e.g., glycosylation and processing e.g., cleavage of protein products can be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to CHO, VERO, BHK, HeLa, COS, MDCK, 293, 3T3, WI38.

For long-term, high-yield production of recombinant proteins, stable expression is preferred. For example, cell lines that stably express the cyclophilin protein can be engineered. Rather than using expression vectors which contain viral origins of replication, host cells can be transformed with DNA controlled by appropriate expression control elements e.g., promoter, enhancer, sequences, transcription terminators, polyadenylation sites, etc., and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci. These foci, in turn, can be cloned and expanded into cell lines. This method can be used to engineer cell lines that express the cyclophilin protein or a cyclophilin-binding protein. Such engineered cell lines can be particularly useful in screening and evaluating of compounds that affect the endogenous activity of the cyclophilin protein.

A number of selection systems can be used, including but not limited to the herpes simplex virus thymidine kinase (Wigler, et al., Cell 11:223, 1977), hypoxanthine-guanine phosphoribosyltransferase (Szybalska & Szybalski, 1962, Proc. Natl. Acad. Sci. USA 48:2026), and adenine phosphoribosyltransferase (Lowy, et al., Cell 22:817, 1980) genes can be employed in tk⁻, hgprt⁻ or aprt⁻ cells, respectively. Also, antimetabolite resistance can be used as the basis of selection for dhfr, which confers resistance to methotrexate (Wigler, et al., Natl. Acad. Sci. USA 77:3567, 1980; O'Hare, et al., Proc. Natl. Acad. Sci. USA 78:1527, 1981); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., J. Mol. Biol. 150:1, 1981); and hygro, which confers resistance to hygromycin genes (Santerre, et al., Gene 30:147, 1984).

Alternatively, any fusion protein may be readily purified by utilizing an antibody specific for the fusion protein being expressed. For example, a system described by Janknecht et al. allows for the ready purification of non-denatured fusion proteins expressed in human cells lines (Janknecht, et al., Proc. Natl. Acad. Sci. USA 88:8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

When used as a component in assay systems such as those described herein, the cyclophilin protein can be labeled, either directly or indirectly, to facilitate detection of a complex formed between the cyclophilin protein and a test substance. Any of a variety of suitable labeling systems can be used including but not limited to radioisotopes such as ¹²⁵I; enzyme labelling systems that generate a detectable colorimetric signal or light when exposed to substrate and fluorescent labels.

Indirect labeling involves the use of a protein, such as a labeled antibody, which specifically binds to either a cyclophilin product or a cyclophilin-binding partner product. Such antibodies include, but are not limited to, polyclonal, monoclonal, chimeric, single chain, Fab fragments and fragments produced by an Fab expression library.

Where recombinant DNA technology is used to produce the cyclophilin protein or a cyclophilin-binding partner, for instance for use in such assay systems, it can be advantageous to engineer fusion proteins that can facilitate labeling (either direct or indirect), immobilization, solubility and/or detection.

Fusion proteins, which can facilitate solubility and/or expression, and can increase the blood half-life of the protein, can include, but are not limited to, soluble Ig-tailed fusion proteins. Methods for engineering such soluble Ig-tailed fusion proteins are well known to those of skill in the art, e.g., U.S. Pat. No. 5,116,964, which is incorporated herein by reference in its entirety. Further, in addition to the Ig-region encoded by the IgG1 vector, the Fc portion of the Ig region utilized can be modified, by amino acid substitutions, to reduce complement activation and Fc binding, e.g., European Patent No. 239400 B1, Aug. 3, 1994.

These and other gene splicing and recombinant protein expression systems well known in the art are useful to produce proteins, such as cyclophilin or a cyclophilin-binding partner protein, and to produce cells that express cyclophilin or cyclophilin-binding partner proteins useful in the context of expression cell engraftment or gene therapy as described herein.

Identifying Therapeutic Targets that Modulate Binding of Cyclophilin to Cyclophilin-Binding Chlamydia Proteins

The following assays are designed to identify compounds or compositions that bind to a cyclophilin polypeptide and its Chlamydia cellular binding partner or partners. Some assays described in this section also allow for the identification of compounds that interfere with the interaction between cyclophilin A and Chlamydia binding proteins.

Compounds may include, but are not limited to, small molecules, peptides such as, e.g., soluble peptides, including but not limited to, peptides comprising portions of cyclophilin A, or the cyclophilin binding domain of the cyclophilin cellular binding partner or receptor, antibodies (including, but not limited to, polyclonal, monoclonal, humanized, anti-idiotypic, chimeric or single chain antibodies, and FAb, F(ab′).sub.2 and FAb expression library fragments, and epitope-binding fragments thereof), and small organic or inorganic molecules. Those compounds identified as inhibitors of the interaction between cyclophilin A and its cellular binding partner or receptor would have utility as anti-Chlamydia agents.

In general, the assays include preparing a reaction mixture of the test compound and cyclophilin and its host receptor or a cellular preparation comprising, in part, the cyclophilin-binding activity and for a time sufficient to allow the components to interact and bind, thus forming a complex which can be removed and/or detected. In order to test a compound for inhibitory activity, the reaction mixture is prepared in the presence and absence of the test compound. The test compound may be initially included in the reaction mixture, or may be added at a time subsequent to the addition of cyclophilin A and its cellular binding partner or receptor. Control reaction mixtures are incubated without the test compound or with a control agent. The formation of any complexes between the target gene protein and the cellular or extracellular binding partner is then detected. The formation of a complex in the control reaction, but not in the reaction mixture containing the test compound, indicates that the compound interferes with the interaction of the target gene protein and the interactive binding partner. Additionally, complex formation within reaction mixtures containing the test compound and normal target gene protein may also be compared to complex formation within reaction mixtures containing the test compound and a mutant target gene protein. This comparison may be important in those cases wherein it is desirable to identify compounds that disrupt interactions of mutant but not normal target gene proteins, to identify mutant target genes and proteins that are themselves, by virtue of their mutations, useful to find their cognate binding partner(s) or receptor(s).

The assay for compounds that interfere with the interaction of the target gene products and binding partners can be conducted in a heterogeneous or homogeneous format. Heterogeneous assays involve anchoring either cyclophilin or putative cyclophilin binding polypeptide, onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance; i.e., by adding the test substance to the reaction mixture prior to or simultaneously with the target gene protein and interactive cellular or extracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the target gene protein or the interactive cellular or extracellular binding partner is anchored onto a solid surface, while the non-anchored species is labeled, either directly or indirectly. Microtitre plates are conveniently utilized. The anchored species may be immobilized by non-covalent or covalent attachments. Non-covalent attachment may be accomplished simply by coating the solid surface with a solution of the target gene product or binding partner, and optionally drying. Alternatively, an immobilized antibody specific for the species to be anchored may be used to anchor the species to the solid surface. The surfaces may be prepared in advance and stored according to procedures well known in the art.

In order to conduct the assay, the partner of the immobilized species is exposed to the coated surface with or without the test compound. After the reaction is complete, unreacted components are removed e.g., by washing and any complexes formed remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

When a homogeneous assay is used, a preformed complex of the target gene protein and the interactive cellular or extracellular binding partner is prepared in which either the target gene product or its binding partners is labeled, but the signal generated by the label is quenched due to complex formation, e.g., U.S. Pat. No. 4,109,496, which utilizes this approach for immunoassays. The addition of a test substance that competes with and displaces one of the species from the preformed complex will result in the generation of a signal above background. In this way, test substances that disrupt target gene protein/cellular or extracellular binding partner interaction can be identified.

In a particular embodiment, the target gene product can be prepared for immobilization using recombinant DNA techniques routinely used in the art. For example, the target gene coding region can be fused to a glutathione-S-transferase (GST) gene using a fusion vector, such as pGEX-5X-1, in such a manner that its binding activity is maintained in the resulting fusion protein. The interactive cellular or extracellular binding partner can be purified and used to raise a monoclonal antibody, using methods routinely practiced in the art. This antibody can be labeled with the radioactive isotope, ¹²⁵I, i.e., by methods routinely practiced in the art. In a heterogeneous assay, e.g., the GST-target gene fusion protein can be anchored to glutathione-agarose beads. The interactive cellular or extracellular binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the target gene protein and the interactive cellular or extracellular binding partner can be detected by measuring the amount of radioactivity that remains associated with the glutathione-agarose beads. Inhibition of the interaction by the test compound will result in a decrease in measured radioactivity.

Alternatively, the GST-target gene fusion protein and the interactive cellular or extracellular binding partner can be mixed together in liquid in the absence of the solid glutathione-agarose beads. The test compound can be added either during or after the species are allowed to interact. This mixture can then be added to the glutathione-agarose beads and unbound material is washed away. Again, the extent of inhibition of the target gene product/binding partner interaction can be detected by adding the labeled antibody and measuring the radioactivity associated with the beads. Such cyclophilin fusion protein constructs are similarly useful to identify and isolate cellular cyclophilin binding partners and mutants thereof, useful in the context of the present invention.

In another embodiment, these same techniques can be employed using peptide fragments that correspond to the binding domains of the target gene protein and/or the interactive cellular or extracellular binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described in this Section above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the for the cellular or extracellular binding partner is obtained, short gene segments can be engineered to express peptide fragments of the protein, which can then be tested for binding activity and purified or synthesized.

For example, and not by way of limitation, a target gene product can be anchored to a solid material as described, above, in this Section by making a GST-target gene fusion protein and allowing it to bind to glutathione agarose beads. The interactive cellular or extracellular binding partner can be labeled with a radioactive isotope, such as ³⁵S, and cleaved with a proteolytic enzyme such as trypsin. Cleavage products can then be added to the anchored GST-target gene fusion protein and allowed to bind. After washing away unbound peptides, labeled bound material, representing the cellular or extracellular binding partner binding domain, can be eluted, purified, and analyzed for amino acid sequence by well-known methods. Peptides so identified can be produced synthetically or fused to appropriate facilitative proteins using recombinant DNA technology.

Therapeutic Agents for Treating Chlamydia Infections

Cyclosporin and Cyclosporin Derivatives

Cyclosporin derivatives that are reported to be devoid of immunosuppressive activity but retain binding capacity for cyclophilin A and inhibit Chlamydia in vitro, have been described. An example is the analog of cyclosporin A, SD2 N1M811, in which the N-methyl-L-leucine unit at position 4 of cyclosporin A is replaced by N-methyl-L-isoleucine. (Traber et al., Antiviral Chemistry & Chemotherapy, 5:331-339, 1994). This cyclosporin analog is reported to be devoid of immunosuppressive activity, but retains full capacity to bind to cyclophilin and exhibit anti-Chlamydia-1 infection activity in vitro as measured by cytopathic effects and particle production. Other derivatized cyclosporin analogs include novel cyclosporin derivatives modified at positions 4 and 5 of cyclosporin A. (EP 484 281, Feb. 24, 1993). Additional immunosuppressive cyclosporin analogs have been developed which consist of cyclosporins with sulfur containing amino acids at position 8. (EP 444 897, Sep. 4, 1991). These cyclosporin derivatives are able to enter the cell, and are thought to mediate their effects intracellularly, after the Chlamydia virus has entered the cell, by targeting the cyclophilin A-Chlamydia interaction.

A cyclosporin derivative can be modified so as to inhibit its entry into or internalization by host cells, such that the derivatives of cyclosporin are correspondingly not interactive with intracellular cyclophilin A. Such cyclosporin derivatives of the present invention are believed to act to inhibit Chlamydia viral entry when presented extracellularly. Such cyclosporin derivatives should inhibit infection by Chlamydia, yet should not result in immunosuppression of the Chlamydia-infected patient. Therefore, the invention encompasses derivatives of cyclosporin which prevent or minimize internalization of the molecule by the cell, yet do not prevent interaction with the cyclophilin, thereby inhibiting Chlamydia-infection. Cyclosporin may be derivatized by the addition of a bulky substituent to the protein. Examples of such substituents, include but are not limited to, charged substituents e.g., spermine or spermidine, polynucleotides with and without modified backbones, carbohydrates e.g., polyacrylic acid, polysodium acrylate, polycesium acrylate, polymethacrylic acid, amphiphilic block copolymers e.g., polystyrene poly sodium acrylate, and amphiphilic homopolymers.

In a preferred embodiment, cyclosporin is derivatized by reaction with polyethylene glycol, resulting in a “pegylated” cyclosporin. Pegylated cyclosporin may be prepared by standard chemical methods known to those skilled in the art. The preferred method of preparing the pegylated cyclosporin of the invention, involves reacting methoxypolyethylene glycol-succinimidyl succinate with 8-amino-cyclosporin A and 4-dimethylamino pyridine in methylene chloride with stirring for two days at room temperature in the dark. To block any unreacted sites, ethanolamine was then added and the mixture incubated at room temperature with stirring for another 24 hours. The derivatized cyclosporin A is purified from the reaction mixture by normal phase HPLC.

Anti-cyclophilin Antibodies

Anti-cyclophilin antibodies can be used as therapeutic agents to treat Chlamydia infections. The anti-cyclophilin antibodies are preferably neutralizing antibodies. If desired, they can be raised against derivatized cyclophilin, in a passive immunotherapy approach. Alternatively, they can be used as a derivatized form as an immunogen to generate an active immune response in Chlamydia-infected patients.

Nucleic Acid-based Therapeutic Agents

Among the compounds that may disrupt the interaction of cyclophilin with its Chlamydia cellular binding partner or receptor are antisense, ribozyme and triple helix molecules. Such molecules are designed to inhibit expression of the target genes cyclophilin A, or its cellular binding partner or receptor in Chlamydia-infected host cells. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred.

Ribozymes are enzymatic RNA molecules capable of catalyzing the specific cleavage of RNA. (For a review, see Rossi, J., Current Biology 4:469-471, 1994). The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins.

Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridize with complementary oligonucleotides, using ribonuclease protection assays.

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarily to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances where the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may be introduced into cells via gene therapy methods such as those described, that do not contain sequences susceptible to whatever antisense, ribozyme, or triple helix treatments are being utilized. Alternatively, in instances whereby the target gene encodes an extracellular protein, it may be preferable to coadminister normal target gene protein in order to maintain the requisite level of target gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

Methods of Treating Chlamydia Infections

Passive Immunotherapy

Neutralizing antibodies which interact with cyclophilin, or with its cellular binding partner can be used to inhibit Chlamydia infection. By way of explanation, but without being limited to any theory, Applicants propose that such antibodies disrupt the interaction between cyclophilin and a Chlamydia host cellular binding partner or receptor, thereby inhibiting Chlamydia-infection. These neutralizing antibodies can be administered in a passive immunotherapy approach. In a preferred embodiment of the invention, the neutralizing antibodies are generated using glycated cyclophilin. Methods for glycating cyclophilin are described in WO 93/23081 which is incorporated herein in its entirety by reference.

Antibodies that are specific for cyclophilin A or its host Chlamydia cellular binding partner or receptor may be generated to inhibit Chlamydia-infection, and these antibodies are thought to work in this regard by interfering with the interaction between virally-associated cyclophilin and its Chlamydia cellular binding partner or receptor. For passive immunotherapy approaches, such antibodies may be generated using standard techniques known to those skilled in the art against the proteins themselves or against peptides corresponding to portions of the proteins. The antibodies include but are not limited to polyclonal, monoclonal, Fab fragments, single chain antibodies, chimeric antibodies, human antibodies, non-human antibodies or humanized antibodies, etc.

Where fragments of the antibody are used, the smallest inhibitory fragment which binds to the target protein's binding domain is preferred. For example, peptides having an amino acid sequence corresponding to the domain of the variable region of the antibody that binds to the target protein may be used. Such peptides can be synthesized chemically or produced via recombinant DNA technology using methods well known in the art.

Alternatively, single chain neutralizing antibodies that bind to intracellular target epitopes may also be administered. Such single chain antibodies may be administered, e.g., by expressing nucleotide sequences encoding single-chain antibodies within the target cell population by utilizing, for example, techniques such as those described in Marasco et al. Proc. Natl. Acad. Sci. USA 90:7889-7893, 1993.

For the production of antibodies, various host animals, may be immunized by injection with the protein, including but not limited to rabbits, mice, rats. Various adjuvants may be used to increase the immunological response, depending on the host species, including but not limited to modification of the antigen of interest by the process of advanced glycation or by advanced glycation products, or by administration of the antigen with Freund's adjuvant (complete and incomplete), mineral gels such as aluminum hydroxide, surface active substances such as lysolecithin, pluronic polyols, polyanions, peptides, oil emersions, key hole limpet hemocyanin, dinitrophenol, and potentially useful human adjuvants such as BCG (bacille Calmette-Guerin) and Corynebacterium parvum. (C. Parker)

Many methods may be used to introduce the passive vaccine formulations described above, these include but are not limited to oral, intradermal, intramuscular, intraperitoneal, intravenous, subcutaneous, and intranasal routes. It may be preferable to introduce the vaccine formulation via the natural route of infection of the pathogen for which the vaccine is designed, or into the tissue where the pathogen resides within the body.

In immunization procedures, the amount of immunogen to be used and the immunization schedule will be determined by a physician skilled in the art and will be administered by reference to the immune response and antibody titers of the subject.

Active Immunotherapy

The present invention further relates to the use of cyclophilin formulated as an immunogen and used as a “vaccine” to generate an active immune response in Chlamydia-infected patients that are not severely immunocompromised or in populations at risk for Chlamydia exposure and infection. In a preferred embodiment, active immunization comprises reacting the immunogen, cyclophilin, with glucose or another reducing sugar under conditions which lead to the formulation of irreversible covalent adducts, known as Advanced Glycation Endproducts (AGEs). Methods for glycating cyclophilin are described in WO 93/23081, which is incorporated herein in its entirety by reference.

An active immune response generated against cyclophilin A, or a cellular binding partner or receptor therefore, which interferes with the interaction between cyclophilin and its cellular binding partner, may be generated to inhibit Chlamydia-infection. For active immunization, a vaccine comprising an immunogen consisting of cyclophilin or a Chlamydia cellular binding partner or receptor, or fragments or peptides having an amino acid sequence corresponding to the domains required for cyclophilin binding to its cellular binding partner or receptor, may be utilized to generate an immune response in the Chlamydia patient or a person at risk for Chlamydia exposure. These immunogens optionally may be modified by advanced glycation or advanced glycation products and may be formulated with an adjuvant to increase the immunological response, such as potentially useful human adjuvants, i.e., BCG (bacille Calmette-Guerin) and Corynebacterium parvum. (C. Parker)

Many methods may be used to introduce the vaccine formulations described above. These include but are not limited to oral, intradermal, intramuscular, intraperitoneal, intravenous, subcutaneous, and intranasal routes. The vaccine formulation may optionally be introduced via the natural route of infection of the pathogen for which the vaccine is designed.

In immunization procedures, the amount of immunogen to be used and the immunization schedule will be determined by a physician skilled in the art and will be administered by reference to the immune response and antibody titers of the subject.

Pharmaceutical Compositions

The therapeutic agents disclosed here can be provided in the form of a pharmaceutical composition. Any suitable administration route and dosage schedule can be used to deliver the inhibitor, or a pharmaceutical composition that includes the inhibitor. When the inhibitor is administered orally, the inhibitor can be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% inhibitor, and preferably from about 25 to 90% inhibitor. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of inhibitor, and preferably from about 1 to 50% inhibitor.

When a therapeutically effective amount of inhibitor is administered by intravenous, cutaneous or subcutaneous injection, the inhibitor will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition inhibitor, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of the inhibitor in the pharmaceutical composition of the present invention will depend upon the nature and severity of the condition being treated, and on the nature of prior treatments which the patient has undergone. Ultimately, the attending physician will decide the amount of inhibitor with which to treat each individual patient. Initially, the attending physician will administer low doses of inhibitor and observe the patient's response. Larger doses of inhibitor may be administered until the optimal therapeutic effect is obtained for the patient, and at that point the dosage is not generally increased further. It is contemplated that the various pharmaceutical compositions used to practice the method of the present invention should contain about 0.1 μg to about 100 mg of inhibitor per kg body weight. Preferred ranges include is a dosages of about 100 μg to 6 mg, and 20 μg to 500 μg per kg of body weight.

The duration of intravenous therapy using the pharmaceutical composition of the present invention will vary, depending on the severity of the disease being treated and the condition and potential idiosyncratic response of each individual patient.

Ultimately the attending physician will decide on the appropriate duration of intravenous therapy using the pharmaceutical composition of the present invention. In some embodiments, the inhibitor is provided in a delayed-release composition. The timed-release composition can include a microparticle.

Microparticles are known in the art and include those described in, e.g., U.S. Pat. No. 6,013,258. Suitable material to include in microparticles includes polymeric material such as poly-lactic-co-glycolic acid (PLGA).

The inhibitor may administered prophylactically or therapeutically and can be administered in conjunction with prophylactic or therapeutic delivery of the vaccine.

If desired the inhibitor can be administered along with a second or additional agents that enhance an immune response. Additional agents include, e.g., adjuvants, cytokines (such as IL-12), and GM-CSF.

The inhibitor can additionally be a nucleic acid encoding an IL-13 inhibitor. Nucleic acids encoding an IL-13 inhibitor can be administered using standard methods, e.g., those described in Feigner et al., U.S. Pat. No., 5,580,859. It is expected that a dosage of approximately 1 to 200 μg of DNA, would be administered per kg of body weight. Where the patient is an adult human, vaccination regimens can include, e.g., intramuscular or subcutaneous administrations of 10-100 μg of nucleic acid when delivered in a microparticle, or of about 100-1000 μg of naked DNA, repeated several times, (e.g., 3 to 6 times).

Other standard delivery methods, e.g., ballistic transfer, or ex vivo treatment, can also be used. In ex vivo treatment, e.g., antigen presenting cells (APCs), dendritic cells, peripheral blood mononuclear cells, or bone marrow cells, can be obtained from a patient or an appropriate donor and activated ex vivo with the immunogenic compositions, and then returned to the patient.

Identifying Agents for Treating Hepatitis C Virus and Dengue Fever Virus

The methods described above for identifying Chlamydia spp. therapeutic agents can also be used to identify agents for treating hepatitis C virus (HCV) infections or infections caused by dengue fever virus. While not wishing to be bound by theory, it is postulated that cyclophilin also associates with HCV and dengue fever virus proteins or proteins, and that this association is necessary for infection and/or propagation of the virus in host cells. Thus, an agent that disrupts the association between cyclophilin and a viral-derived polypeptide can be used as a therapeutic agent, or a target for identifying agent, for treating these viral infections.

One way to identify a therapeutic agent for treating HCV or dengue fever virus is to examine a test agent for its ability to bind to cyclophilin. These agents are identified by providing a cyclophilin polypeptide, contacting the cyclophilin polypeptide with a test agent, and determining whether the test agent binds the cyclophilin polypeptide. Binding of the test agent to the cyclophilin polypeptide indicates the test agent is a therapeutic target for treating a Hepatitis C virus or dengue fever virus infection.

The cyclophilin is preferably provided as a substantially purified cyclophilin polypeptide. Any cyclophilin polypeptide known in the art can be used, e.g., cyclophilin A, cyclophilin B, cyclophilin C, or cyclophilin D. As used herein, “substantially pure” polypeptide means a polypeptide separated from components that naturally accompany it. Typically, the polypeptide is substantially pure when it is at least 60%, by weight, free from the proteins and naturally-occurring organic molecules with which it is naturally associated. Preferably, the purity of the preparation is at least 75%, more preferably at least 90%, and most preferably at least 99%, by weight. A substantially pure cyclophilin polypeptide fragment can be obtained, for example, by extraction from a natural source, by expression of a recombinant nucleic acid encoding a cyclophilin polypeptide fragment, by expression of a cyclophilin polypeptide fragment fusion protein, or by chemical synthesis. A chemically synthesized polypeptide or a polypeptide produced in a cellular system different from the cell from which it naturally occurs is, by definition, substantially free from components that naturally accompany it. Accordingly, substantially pure polypeptides include those derived from eukaryotic organisms but synthesized in recombinant cells of E. coli or other prokaryotes. Purity can be measured by any appropriate methods, e.g., column chromatography, polyacrylamide gel electrophoresis, or by HPLC analysis.

The cyclophilin polypeptide can be provided as a labeled polypeptide. The label can be, e.g., a radioactive label or a non-radioactive label such as a fluorescent label. Alternatively, the cyclophilin can be labeled with a label such as biotin.

In some embodiments, the cyclophilin polypeptide is provided attached to a substrate. The substrate may include a plurality of cyclophilin polypeptides, e.g., one or more of cyclophilin A, cyclophilin B, cyclophilin C, cyclophilin D, or a mixture of these polypeptides. The cyclophilin polypeptides are provided on the substrate at one or more addressable locations.

The substrate can be, e.g., a planar surface or a spherical surface (such as a bead).

The method can also be performed by providing a cyclophilin polypeptide that is associated with a cell. The cyclophilin polypeptide can be provided as a labeled polypeptide. The label can be, e.g., a radioactive label or a non-radioactive label such as a fluorescent label. Alternatively, the cyclophilin can be labeled with a label such as biotin.

A therapeutic agent can also be identified by providing a sample that includes a Hepatitis C virus or dengue fever virus polypeptide and contacting the sample with a cyclophilin polypeptide under conditions allowing for formation of a complex between at least one Hepatitis C virus or dengue fever virus protein in the sample and the cyclophilin polypeptide. The complex is then detected, and at least one Hepatitis C virus or dengue fever virus protein in the complex is identified. The identified Hepatitis C virus or dengue fever virus protein(s) are useful, e.g., as a target for identifying therapeutic agents for treating hepatitis associated with an HCV infection or dengue fever. A cyclophilin-binding polypeptide can be used to raise antibodies that can, in turn, be used to treat Hepatitis C or dengue fever virus infections. In some embodiments, the complex is detected with an anti-cyclophilin antibody.

Another alternative for identifying a therapeutic agent for treating a Hepatitis C virus or dengue fever virus infection by providing a sample that includes a Hepatitis C virus or dengue fever virus polypeptide and a cyclophilin polypeptide and contacting the sample with a cyclophilin probe under conditions that allow for formation of a complex between the cyclophilin probe and the Hepatitis C virus or dengue fever virus protein. The complex is detected and the Hepatitis C virus or dengue fever virus protein in the complex is identified. The identified Hepatitis C virus or dengue fever virus protein can be used, e.g., as a therapeutic target for treating a Hepatitis C virus or dengue fever virus infection. For example, antibodies against the cyclophilin-binding Hepatitis C virus or dengue fever virus protein can be raised and test for their efficacy in treating Hepatitis C virus or dengue fever virus infection.

In some embodiments, the Hepatitis C virus or dengue fever virus proteins are labeled, e.g., with biotin.

The invention additionally provides a method of identifying a therapeutic agent for treating Hepatitis C virus or dengue fever virus infections by providing a cell infected with Hepatitis C virus or dengue fever virus, contacting the cell with an agent that inhibits cyclophilin, and determining whether the agent inhibits the pathogenicity of the virus. Inhibition of pathogenicity of the Hepatitis C virus or dengue fever virus indicates the agent is a therapeutic agent for treating Hepatitis C virus or dengue fever virus.

In a further aspect, the invention provides a method of identifying an agent that inhibits infection of a eukaryotic host cell by a Hepatitis C virus or dengue fever virus by providing a cell infected with Hepatitis C virus or dengue fever virus, contacting the cell with an agent that inhibits at least one activity of a cyclophilin polypeptide; and determining whether the agent inhibits infection by Hepatitis C virus or dengue fever virus.

Another screening method for identifying compounds capable of interfering with the formation of a complex between a cell infected with Hepatitis C virus or dengue fever virus and a cyclophilin polypeptide includes: (a) producing a cyclophilin affinity fusion protein; (b) preincubating a compound with the cyclophilin affinity fusion protein of step (a); (c) adding a cell infected with Hepatitis C virus or dengue fever virus to the incubate of step (b) under conditions which permit one or more Hepatitis C virus or dengue fever virus proteins and the cyclophilin affinity fusion protein to form a complex; (d) contacting the incubate of step (c) with an affinity medium under conditions that allow the Hepatitis C virus or dengue fever virus-cyclophilin affinity fusion protein complex to bind to the affinity medium; (e) determining the amount of the Hepatitis C virus or dengue fever virus-cyclophilin affinity fusion protein complex formation by comparison to a control sample lacking the compound. Reduced binding of Hepatitis C virus or dengue fever virus protein to the cyclophilin affinity fusion protein indicates the compound inhibits formation of the complex. The cyclophilin fusion polypeptide can be, e.g., cyclophilin A, cyclophilin B, cyclophilin C, or cyclophilin D. In some embodiments, the cyclophilin affinity fusion protein is a glutathione S-transferase-cyclophilin (GST-cyclophilin) fusion protein and/or the affinity medium includes glutathione-agarose beads.

The compound can alternatively be identified by a method includes:(a) producing a Hepatitis C virus or dengue fever virus affinity fusion protein; (b) preincubating a compound with the Hepatitis C virus or dengue fever virus affinity fusion protein of step (a); (c) adding a cyclophilin to the incubate of step (b) under conditions which permit the cyclophilin and the Hepatitis C virus or dengue fever virus affinity fusion protein to form a complex; (d) contacting the incubate of step (c) with an affinity medium under conditions that enable the cyclophilin-Hepatitis C virus or dengue fever virus fusion protein complex to bind the affinity medium; and (e) determining the amount of the cyclophilin-Hepatitis C virus or dengue fever virus affinity fusion protein complex formation by comparison to a control sample lacking the compound. Reduced binding indicates the compound inhibits cyclophilin-Hepatitis C virus or dengue fever virus affinity fusion protein complex formation. The cyclophilin polypeptide can be, e.g., cyclophilin A, cyclophilin B, cyclophilin C, and cyclophilin D, or combinations thereof. In some embodiments, the cyclophilin affinity fusion protein is a glutathione S-transferase-cyclophilin (GST-cyclophilin) fusion protein and/or the affinity medium includes glutathione-agarose beads.

The cyclophilin can be labeled if desired with, e.g., a fluorescent label, a radioactive label, or a chemiluminescent label.

Suitable HCV proteins for use in these assays include, e.g., the HCV core, E1, E2, P7, NS2, NS3, NS4A, NS4B, NS5A, and NS5B polypeptides (see McHucthison et al., Hepatology 36:S246-52, 2002). To perform cell-based assays, primary cultures of human hepatocytes can be used, although the cells do not support the production of progeny virions (Fournier et al., J. Gen. Virol. 79:2367-74, 1998). Other sources for cell-based assays included cells from HCV-infected human hepatocytes transplanted into the liver of immunodeficient mice can be used (see Bronowicki et al. Hepatology 28:211-18, 1998 and Lerta et al. Hepatology 28:498A (1998). Alternatively, cells from tree shrews can be used, as these animals are susceptible for infection with human HCV (Xie et al. Virology 244:513-20, 1998).

The agents identified above can be used to treat infections caused by hepatitis C dengue fever. For example, a Hepatitis C virus or dengue fever virus infection can be treated by administering to a subject by a therapeutically effective amount of an agent that inhibits activity of cyclophilin. In some embodiments, the agent is a polypeptide that includes a cyclophilin-binding region of an anti-cyclophilin antibody polypeptide. An example of such an agent is an anti-cyclophilin antibody. The antibody can be, e.g., a polyclonal antibody or a monoclonal antibody. The subject can be, e.g., a human, a non-human primate (such as a chimpanzee, gorilla, or new world monkey), cow, horse, pig, goat, sheep, dog, cat, or bird.

In other embodiments, the agent is an antibiotic that inhibits the activity of a cyclophilin polypeptide. An example of such an antibiotic is cyclosporin. The cyclosporin can be provided as a cyclosporin derivative.

The invention will be further illustrated in the following examples, which do not limit the scope of the appended claims.

### EXAMPLE 1

### Identification of Cyclophilin and Cyclophilin-Binding Chlamydia Polypeptides in SDS-PAGE Preparations

Elemental bodies (EB) from C. pneumoniae and C. trachomatis were analyzed by SDS-PAGE and transferred to a nitrocellulose blot in duplicate. Both blots were blocked with BSA and washed with PBS. One blot was incubated with 200 μg of cyclophilin A protein. The other blot was incubated only with PBS. Both blots were then washed and treated with anti-cyclophilin A antibody followed by a secondary anti rabbit AP conjugated antibody. The blots were developed with a Biorad AP kit.

A 30 kD band of 30 kD was present in the C. pneumoniae EBs in the blot treated with cyclophilin A. This band was absent from the blot treated with only PBS.

For the C. trachomatis EBs, an intense 30 kD band was recognized in the blot treated with cypA. This band was absent from the blot treated with only PBS. In addition, a doublet between 50 and 70 kD was recognized in the cyclophilin treated blot for C. trachomatis elementary bodies. This band was very weakly recognized in the blots treated with only PBS.

In a addition, some high molecular weight bands of about 100 kD were detected in the blot treated with cyclophilin A that were not present in the untreated blot.

The presence of cyclophilin A in the elementary body preparations was also examined using an anti-cyclophilin A antibody. Cyclophilin A was present in all the elementary body preparations from both C. trachomatis and C. pneumonie.

### EXAMPLE 2

### Identification of Cyclophilin-Binding Polypeptides in Chlamydia EBs Using Cyclophilin Immobilized to Magnetic Beads

The presence of cyclophilin-binding polypeptides in C. pneumoniae EBs was examined using M280 magnetic beads (Dynal Biotech, Inc., Lake Success, N.Y.) coated with recombinant cyclophilin A. These beads were incubated with proteins from a lysate of surface biotinylated EBs.

Bound protein was characterized further using western blot hybridization analysis. The Chlamydia major outer membrane protein (MOMP) was identified using an anti MOMP mouse polyclonal antibodies

### EXAMPLE 3

### Identification of Cyclophilin-Binding Polypeptides in Chlamydia Cell Fractions Using Cross-Linking Agents

An elementary body lysate and cycophilin A are mixed in the presence in the presence of the crosslinker BS3. Crosslinked complexes of the cyclophilin A polypeptide and one or more Chlamydiae proteins are recovered, and the identity of the Chlamydia protein or proteins determined using antibodies to Chlamydia proteins.

### EXAMPLE 4

### Identification of Chlamydia Proteins that Bind to Cyclophilin Proteins Immobilized on a Substrate

Cyclophilin-binding Chlamydia polypeptides were identified by identifying Chlamydiae proteins that are able to bind to cyclophilin polypeptides immobilized on a solid substrate.

Cyclophilin A was dialyzed into PBS and coupled to a glass slide covered with a deposited 50 nm layer of gold (Biacore, Inc., Piscataway, N.J.). Binding of some recombinant Chlamydia proteins to the cyclophilin A immobilized on the chip was determined using surface plasmon resonance (SPR).

The C. pneumoniae protein T776, which is about 76 kD, was identified. To verify this interaction, the T776 protein was bound to the chip and its ability to bind Cyclophilin A was determined. Binding to the T776 protein was detected. No binding to a glass slide coated with BSA was observed in the same conditions.

### EXAMPLE 5

### Antibodies to Cyclophilin Block Chlamydia Infection of Human Cells

The ability of anti cyclophilin antibodies to block Chlamydia infection of human cells was examined.

A 1:4 diluted rabbit polyclonal antibody to cyclophilin A, (CalBiochem, Cat #239778; and Cat #06-512 from Upstate Biotechnology Inc., (Waltham, Mass.) neutralized Chlamydia at a titer of 160 and 640 respectively in vitro. These antibodies did not cross react to recombinant macrophage infectivity potentiator (MIP) polypeptide from Chlamydia pneumoniae. MIP, like cyclophilin, is a peptidyl prolyl cis-trans isomerase enzymes. Polyclonal mouse antisera to Chlamydia MIP protein did not recognize or very faintly react with cyclophilin A.

### OTHER EMBODIMENTS

Other aspects, advantages, and modifications are within the scope of the following claims.

What is claimed is:

1. A method of identifying a therapeutic agent for treating a Chlamydia spp. infection, the method comprising:
providing a cyclophilin polypeptide;
contacting said cyclophilin polypeptide with a test agent; and
determining whether said test agent binds said cyclophilin polypeptide,
wherein binding of said test agent to said cyclophilin polypeptide indicates said test agent is a therapeutic agent for treating a Chlamydia spp. infection.

2. The method of claim 1, wherein said cyclophilin polypeptide is provided as a substantially purified cyclophilin polypeptide.

3. The method of claim 1, wherein said cyclophilin polypeptide is cyclophilin A, cyclophilin B, cyclophilin C, or cyclophilin D.

4. The method of claim 1, wherein said cyclophilin polypeptide includes a label.

5. The method of claim 4, wherein said label is biotin.

6. The method of claim 2, wherein said cyclophilin polypeptide is provided attached to a substrate.

7. The method of claim 6, wherein said substrate comprises a plurality of cyclophilin polypeptides.

8. The method of claim 7, wherein said substrate comprises one or more of cyclophilin A, cyclophilin B, cyclophilin C, cyclophilin D, or a mixture thereof.

9. The method of claim 6, wherein said cyclophilin polypeptide is provided on said substrate at one or more addressable locations.

10. The method of claim 6, wherein said substrate is a planar surface.

11. The method of claim 6, wherein said substrate is a bead.

12. The method of claim 1, wherein said cyclophilin polypeptide is provided associated with a Chlamydia cell.

13. The method of claim 12, wherein said cyclophilin polypeptide includes a label.

14. The method of claim 13, wherein said label is biotin.

15. The method of claim 12, wherein said association is by binding of said cyclophilin polypeptide to a Chlamydia polypeptide.

16. The method of claim 12, wherein said cyclophilin is provided in association with a Chlamydia elementary body.

17. The method of claim 16, wherein said association of cyclophilin and Chlamydia cell is by binding of said cyclophilin polypeptide to a Chlamydia polypeptide.

18. The method of claim 16, wherein said Chlamydia cell is a Chlamydia trachomatis cell.

19. The method of claim 16, wherein said Chlamydia cell is a Chlamydia pneumoniae cell.

20. A method for identifying a cyclophilin-binding Chlamydia polypeptide, the method comprising:
providing a sample comprising a Chlamydia polypeptide;
contacting said sample with a cyclophilin polypeptide under conditions allowing for formation of a complex between at least one Chlamydia protein in said sample and said cyclophilin polypeptide;
detecting said complex; and
identifying said at least one cyclophilin-binding Chlamydia polypeptide in said complex.

21. The method of claim 20, wherein said complex is detected with an anti-cyclophilin antibody.

22. The method of claim 21, wherein said complex is detected with a monoclonal anti-cyclophilin antibody.

23. A method for identifying a therapeutic agent for treating a Chlamydia infection, the method comprising
providing a sample comprising a Chlamydia polypeptide and a cyclophilin polypeptide;
contacting said sample with a cyclophilin probe under conditions that allow for formation of a complex between said cyclophilin probe and said Chlamydia polypeptide;
detecting said complex; and
identifying said Chlamydia polypeptide in said complex,
thereby identifying a therapeutic agent for treating a Chlamydia infection.

24. The method of claim 23, wherein said Chlamydia polypeptide includes a label.

25. The method of claim 24, wherein said label is biotin.

26. The method of claim 23, wherein said cyclophilin probe is an anti-cyclophilin antibody.

27. A purified complex of a cyclophilin polypeptide and a Chlamydia protein selected from the group consisting of a T776 polypeptide, 30 kD polypeptide, a 40 kDa polypeptide, and a Chlamydia major outer membrane protein (MOMP).

28. A method of identifying a therapeutic agent for treating a Chlamydia infection, the method comprising:
providing a Chlamydia cell;
contacting said cell with an agent that inhibits at least one activity of a cyclophilin polypeptide; and
determining whether said agent inhibits the pathogenicity of said Chlamydia cell,
wherein inhibition of pathogenicity of said Chlamydia cell indicates said agent is a therapeutic agent for treating Chlamydia.

29. A method of identifying an agent that inhibits infection of a eukaryotic host cell by a Chlamydia cell, the method comprising
providing a Chlamydia cell;
contacting said cell with an agent that inhibits at least one activity of a cyclophilin polypeptide; and
determining whether said agent inhibits infection of said Chlamydia cell.

30. A method for identifying a compound that interferes with the formation of a complex between a Chlamydia cell and a cyclophilin polypeptide, the method comprising:
(a) producing a cyclophilin affinity fusion protein;
(b) preincubating a compound with the cyclophilin affinity fusion protein of step (a);
(c) adding a Chlamydia sample to the incubate of step (b) under conditions which permit Chlamydia and the cyclophilin affinity fusion protein to form a complex;
(d) contacting the incubate of step (c) with an affinity medium under conditions that allow the Chlamydia-cyclophilin affinity fusion protein complex to bind to said affinity medium;
(e) determining the amount of said Chlamydia-cyclophilin affinity fusion protein complex formation by comparison to a control sample lacking said compound;
wherein reduced binding of Chlamydia to the cyclophilin affinity fusion protein is indicative of the ability of said compound to inhibit said complex formation.

31. The method of claim 30, wherein the cyclophilin in said cyclophilin fusion polypeptide is selected from the group consisting of cyclophilin A, cyclophilin B, cyclophilin C, and cyclophilin D.

32. The method of claim 30, wherein the cyclophilin affinity fusion protein is a glutathione S-transferase-cyclophilin (GST-cyclophilin) fusion protein.

33. The method of claim 30, wherein the affinity medium comprises glutathione-agarose beads.

34. A method for identifying a compound capable of interfering with the formation of a complex between a cyclophilin polypeptide and a Chlamydia affinity fusion protein, the method comprising:
(a) producing a Chlamydia affinity fusion protein;
(b) preincubating a compound with the Chlamydia affinity fusion protein of step (a);
(c) adding a cyclophilin polypeptide to the incubate of step (b) under conditions which permit the cyclophilin and the Chlamydia affinity fusion protein to form a complex;
(d) contacting the incubate of step (c) with an affinity medium under conditions that enable the cyclophilin-Chlamydia fusion protein complex to bind said affinity medium;
(e) determining the amount of said cyclophilin-Chlamydia affinity fusion protein complex formation by comparison to a control sample lacking said compound;
wherein reduced binding indicates said compound inhibits cyclophilin-Chlamydia affinity fusion protein complex formation.

35. The method of claim 34, wherein the cyclophilin employed is selected from the group consisting of cyclophilin A, cyclophilin B, cyclophilin C, and cyclophilin D.

36. The method of claim 34, wherein the affinity medium comprises glutathione-agarose beads.

37. The method of claim 34, wherein the cyclophilin is labeled with a label selected from the group consisting of a fluorescent label, a radioactive label, and a chemiluminescent label.
